(19) United States
(12) Patent Application Publication
(10) Pub. No.: US 2023/0404414 A1
(43) Pub. Date: Dec. 21, 2023

(54) AI-enhanced Wearable Photo-Electro-Tonoarteriography (PETAG) Method And Apparatus

(71) Applicant: Hong Kong Centre for Cerebro-Cardiovasculer Health Engineering Limited, Hong Kong (CN)

(72) Inventors: Yuanting ZHANG, Hong Kong (CN); Ting XIANG, Hong Kong (CN); Nan JI, Hong Kong (CN); David A. CLIFTON, Oxford (GB); Lei LU, Oxford (GB); Tingting ZHU, Oxford (GB)

(21) Appl. No.: 17/981,362

(22) Filed: Nov. 4, 2022

(30) Foreign Application Priority Data
Jun. 21, 2022 (CN) 202210706973.1

(51) Int. Cl. A61B 5/021; A61B 5/25; A61B 5/332

(52) U.S. Cl. CPC A61B 5/021; A61B 5/25; A61B 5/332

(57) ABSTRACT

The present invention relates to an AI-enhanced wearable photo-electro-tonoarteriography (PETAG) method and apparatus. The invention relates to the technical fields of medical detection and artificial intelligence, and is applicable to, such as, tonoarteriogram (TAG) signal estimation, which is continuous blood pressure and cardiac diseases detection. The method comprises: acquiring at least one lead electrocardiogram (ECG) signal and multi-wavelength photoplethysmogram signals (MWPPG signals); processing the ECG signal and the MWPPG signals by a multimodal model-based multi-task learning network, determining a signal processing result related to a TAG information and/or related to a cardiac disease information. The present invention is advantageous in reducing computational cost involved in signal processing on the basis of ensuring accuracy.

### FIELD OF THE INVENTION

The present invention relates to the technical field of medical testing and artificial intelligence. Specifically, the invention relates to an AI-enhanced wearable photo-electro-tonoarteriography (PETAG) method and apparatus.

### BACKGROUND OF THE INVENTION

According to the World Health Organization (WHO), cardiovascular disease (CVD) is the leading cause of death worldwide, accounting for approximately 32% of all deaths, and is expected to increase to more than 23 million by 2030. Hypertension, the highest risk factor for death, affects approximately 1.28 billion adults worldwide, and more than 700 million people have untreated hypertension. Hypertension exists if systolic blood pressure is consistently at or above 140 mmHg or if diastolic blood pressure is maintained at or above 90 mmHg. If the systolic pressure is between 120 and 139 mmHg or the diastolic pressure is between 80 and 89 mm Hg, the person is prehypertensive. A survey data shows that the prevalence of hypertension among adults in China is 23.2%, and the number of people with the disease is 245 million. In other words, 1 in 4 adults suffer from hypertension. At the same time, the “reserve army” of hypertension is also rushing in. The prevalence of pre-hypertension is as high as 435 million people. This is equivalent to 1 in 2 adults being pre-hypertensive. In addition, several studies have reported that many electrocardiogram (ECG) abnormalities are associated with an increased risk of death from CVD and coronary heart disease (CHD). Early detection of CVD is important. As two major risk factors/indicators of CVD, hypertension and ECG abnormalities, the need for unperturbed monitoring of blood pressure and cardiac status in daily life is increasing significantly.

ECG records the changes in electrical activity produced by the heart during each cardiac cycle from the body surface and has been widely used to diagnose and monitor abnormal cardiac conditions such as atrial fibrillation, ventricular fibrillation, myocardial infarction, etc. Currently, due to the increasing demand for monitoring personal health and the development of wearable sensors in healthcare and mobile devices, there is a desire to detect and monitor personal health conditions unobtrusively, especially blood pressure monitoring, screening, and ECG abnormality detection at home and in the community.

Several wearable devices are already available in the market for ECG signal monitoring, heart rate/heart rate variability and arrhythmia detection, such as sports T-shirts, sports underwear, and watches. However, the existing devices collect limited signals and are difficult to do further signal analysis and processing.

### OBJECTS OF THE INVENTION

An object of the present invention is to provide an AI-enhanced wearable photo-electro-tonoarteriography (PETAG) method and apparatus.

The above object is met by the combination of features of the main claims; the sub-claims disclose further advantageous embodiments of the invention.

One skilled in the art will derive from the following description other objects of the invention. Therefore, the foregoing statements of object are not exhaustive and serve merely to illustrate some of the many objects of the present invention.

### SUMMARY OF THE INVENTION

Embodiments of the present invention provide an AI-enhanced wearable photo-electro-tonoarteriography (PETAG) method and apparatus to solve at least one of the above technical problems.

In a first aspect, embodiments of the present invention provide an AI-enhanced wearable photo-electro-tonoarteriography (PETAG) method comprising: acquiring at least one lead ECG signal and multi-wavelength photoplethysmogram (MWPPG) signals; processing the ECG signal and the MWPPG signals by a multimodal model-based multi-task learning network, determining a signal processing result related to a tonoarteriogram (TAG) information and/or related to a cardiac disease information.

In a possible embodiment, wherein the step of acquiring at least one lead EGC signal and MWPPG signals comprises: acquiring by collecting at least one lead ECG signal and MWPPG signals from a clothing worn by a target subject; wherein cloth of said clothing is provided with an ECG electrode and a multi-wavelength photoplethysmography sensors.

In a possible embodiment, wherein the ECG electrode and the multi-wavelength photoplethysmography sensors are arranged at the clothing using one of the following: manufacturing the ECG electrode and the multi-wavelength photoplethysmography sensors at a chest portion and a waist portion of a detachable tight belt, with the detachable tight belt being fixed at a corresponding position of an electric conductive clothing; manufacturing the ECG electrode and the multi-wavelength photoplethysmography sensors at a vest and/or a waistband combined with the electric conductive clothing; manufacturing the ECG electrode and the multi-wavelength photoplethysmography sensors at a modified tight clothing.

In a possible embodiment, wherein the EGC electrode is made of at least one of electronic fabric materials, ionic hydrogels and other soft electric conductive materials; the multi-wavelength photoplethysmography sensors is integrated in the ECG electrode.

In a possible embodiment, wherein before processing the ECG signal and the MWPPG signals by a multimodal model-based multi-task learning network, further comprising: filtering noise from the ECG signal and the MWPPG signals to obtain a noise reduced signal; converting the noise reduced signal to obtain a noise reduced ECG signal and MWPPG signals of different leads.

In a possible embodiment, wherein the filtering noise from the ECG signal and the MWPPG signals to obtain the noise reduced signal comprises: iteratively performing the following operations until a stop iteration condition is met: combining impedance information between electrode of a left arm and a right arm and between electrode of a right arm and a left leg, filtering noise from the ECG signal and the MWPPG signals, and generating a current noise reduced signal; and using the noise reduced signal and the impedance information as input data for a next iteration, filtering the noise from the ECG signal and the MWPPG signals.

In a possible embodiment, wherein the step of processing the ECG signal and the multi-wavelength signal by a multimodal model-based multi-task learning network, determining a signal processing result related to a TAG information and/or related to a cardiac disease information, comprising: performing feature extraction on the ECG signal and the MWPPG signals through frequency domain attention based neural network and time domain interpretative based neural network to obtain a first feature information; performing feature extraction on the MWPPG signals to obtain a second feature information; performing pooling operation on the first feature information; performing feature fusion classification process against the second feature information and the pooled first feature information to determine the signal processing result related to the TAG information and/or related to the cardiac disease information.

In a possible embodiment, wherein the signal processing result related to the TAG information comprises at least one of a TAG signal, a systolic blood pressure information, a diastolic blood pressure information, a blood pressure variation (BPV) information and a hypertension information; the signal processing result related to the cardiac disease information comprises at least one of an electrocardiogram, an arrhythmia detection result and a myocardial infarction detection result.

In a possible embodiment, further comprising at least one of the following: transmitting the signal processing result to a user apparatus to display the signal processing result to a user; the user apparatus comprises at least one of a mobile phone, a watch and glasses; uploading the signal processing result to a cloud database and/or a medical platform.

In a second aspect, embodiments of the present invention provide an AI-enhanced wearable photo-electro-tonoarteriography (PETAG) apparatus, comprising: an acquisition module for acquiring at least one lead ECG signal and MWPPG signals; a processing module for processing the ECG signal and the MWPPG signals by a multimodal model-based multi-task learning network, determining a signal processing result related to a TAG information and/or related to a cardiac disease information.

In a third aspect, embodiments of the present invention provide an electronic apparatus, comprising: a memory, a processor and a computer program stored in the memory, wherein the processor executes the computer program to implement the method steps of the AI-enhanced wearable photo-electro-tonoarteriography (PETAG) in the first aspect.

In a fourth aspect, embodiments of the present invention provide a computer-readable storage medium on which a computer program is stored, wherein, the computer program is executed by a processor to implement the method steps of the AI-enhanced wearable photo-electro-tonoarteriography (PETAG) in the first aspect.

In a fifth aspect, embodiments of the present invention provide a computer program product, comprising a computer program, wherein, the computer program is executed by a processor to implement the method steps of the AI-enhanced wearable photo-electro-tonoarteriography (PETAG) in the first aspect.

In a sixth aspect, embodiments of the present invention provide an AI-enhanced wearable photo-electro-tonoarteriography (PETAG) system, comprising the said clothing and electronic devices designed with sensing module and communication module; acquiring by collecting at least one lead ECG signal and MWPPG signals from the sensing module in the clothing, and transmitting the obtained ECG signal and MWPPG signals to the said electronic devices through the communication module; the said electronic devices implement the method steps of the AI-enhanced wearable photo-electro-tonoarteriography (PETAG) in the first aspect.

In a possible embodiment, the system characterized in that said sensing module comprises an ECG electrode and a multi-wavelength photoplethysmography sensors.

The present invention embodiment provides an AI-enhanced wearable photo-electro-tonoarteriography (PETAG) method and apparatus; specifically, after acquiring specific lead electrocardiosignal (ECG signal) and MWPPG signals, they can be input to a multimodal model-based multi-task learning network for signal processing, the signal processing results related to TAG information and/or related to heart disease information are determined; the implementation of the present invention can save computational costs and reduce the complexity of manufacturing detection devices related to blood pressure on the basis of ensuring accuracy.

### BRIEF DESCRIPTION OF THE DRAWINGS

FIG. 1 shows a schematic diagram of the AI-enhanced wearable photo-electro-tonoarteriographic (PETAG) method;

FIG. 2 shows different configurations of ECG electrodes and MWPPG sensors of the clothing;

FIG. 3 shows different configurations of 12-lead ECG electrodes and MWPPG sensors of the clothing;

FIG. 4 shows a structure diagram of the proposed PETAG system;

FIG. 5 shows another structure diagram of the proposed PETAG system;

FIG. 6 shows another structure diagram of the proposed PETAG system;

FIG. 7 shows a diagram of adaptive filter;

FIG. 8 shows an overall framework of the proposed MMM-net model;

FIG. 9 shows a schematic diagram of the wireless communication of external devices;

FIG. 10 shows a structure diagram of the proposed PETAG apparatus; and

FIG. 11 shows a schematic diagram of a structure of an electronic device.

### DESCRIPTION OF PREFERRED EMBODIMENTS

The present invention relates to the technical field of medical testing and artificial intelligence, specifically, the invention relates to an AI-enhanced wearable photo-electro-tonoarteriography (PETAG) method and apparatus.

Embodiments of the present invention are described below in connection with the accompanying drawings in the present invention. It should be understood that the embodiments set forth below in connection with the accompanying drawings are exemplary descriptions for the purpose of explaining the technical solutions of the embodiments of the present invention and do not constitute a limitation of the technical solutions of the embodiments of the present invention.

It will be understood by those skilled in the art that, unless specifically stated, the singular forms “one”, “a”, “said” and “the” used herein” may also include the plural form. It should be further understood that the terms “includes” and “comprises” as used in embodiments of this invention mean that the corresponding features may be implemented as the features, information, data, steps, operations, components and/or assemblies presented, but do not exclude the implementation of other features, information, data, operations, components and/or assemblies that are supported in the art. It is to be understood that when we refer to a component as a feature, information, data, step, operation, element, component, and/or assembly, it is to be understood that it is to be implemented as the feature, information, data, step, operation, component, and/or assembly presented. It should be understood that when we refer to a component being “connected” or “coupled” to another component, the component may be directly connected or coupled to the other component, or it may refer to the component and the other component being connected through an intermediate component. In addition, the “connection” or “coupling” as used herein may include wireless connection or wireless coupling. The term “and/or” as used herein indicates at least one of the items defined by the term, for example, “A and/or B” may be implemented as “A”, or as “B”, or “A and B”.

In order to make the purpose, technical solutions and advantages of the present invention clearer, the embodiments are described in further detail below in conjunction with the accompanying drawings.

The present invention relate to Artificial Intelligence (AI) is the theory, methods, techniques and invention systems that use digital computers or machines controlled by digital computers to simulate, extend and expand human intelligence, perceive the environment, acquire knowledge and use the knowledge to obtain optimal results. In other words, artificial intelligence is an integrated technology of computer science that attempts to understand the essence of intelligence and to produce a new intelligent machine that can respond in a similar way to human intelligence. Artificial intelligence is also the study of the design principles and implementation methods of various intelligent machines to make them capable of perception, reasoning and decision making. Artificial intelligence technology is a comprehensive discipline that covers a wide range of fields, both at the hardware and software levels. Basic AI technologies generally include technologies such as sensors, special AI chips, cloud computing, distributed storage, big data processing technologies, operation/interaction systems, mechatronics, etc. Artificial intelligence software technologies mainly include computer vision technology, speech processing technology, natural language processing technology, and machine learning/deep learning, autonomous driving, intelligent transportation, and other major directions.

The AI-enhanced wearable photo-electro-tonoarteriography (PETAG) method proposed in this invention specifically relates to Machine Learning (ML), which is a multi-disciplinary intersection involving probability theory, statistics, approximation theory, convex analysis, algorithmic complexity theory, and many other disciplines. It specializes in studying how computers can simulate or implement human learning behaviors to acquire new knowledge or skills and reorganize existing knowledge structures to continuously improve their performance. Machine learning is the core of artificial intelligence and is the fundamental way to make computers intelligent, and its applications span all areas of artificial intelligence. Machine learning and deep learning usually include techniques such as artificial neural networks, confidence networks, reinforcement learning, transfer learning, inductive learning, and style teaching learning. For example, the tonoarteriogram (TAG) signal can be estimated by machine learning based models for the acquired signal, etc.

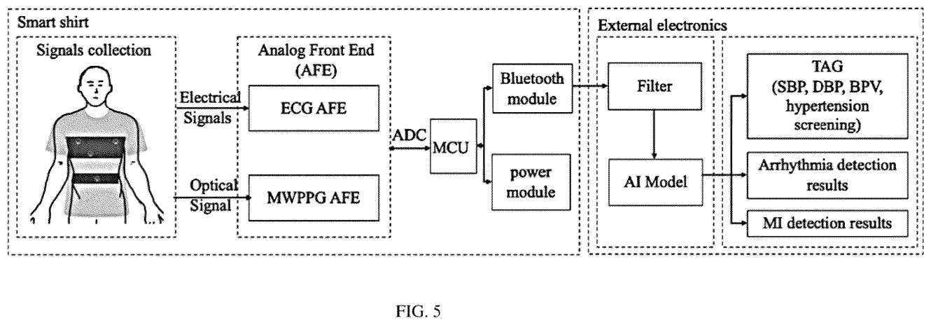

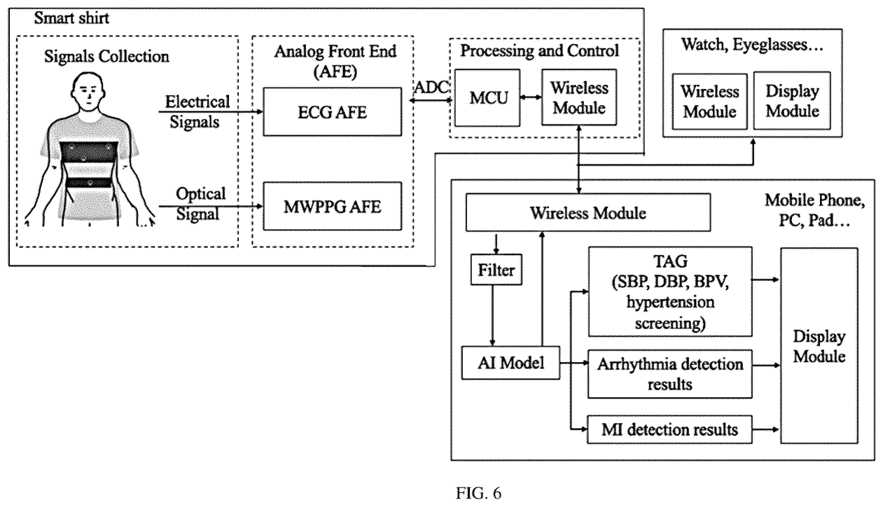

This embodiment provides an AI-enhanced wearable photo-electro-tonoarteriography (PETAG) method in its operating environment, which may include a terminal, a server.

Wherein, the terminal may run a client or a service platform. The terminal (which may also be referred to as a device) may be a smartphone, a tablet, a laptop, a desktop computer, a smart voice interaction device (e.g., a smart speaker), a wearable electronic device (e.g., a smart watch), an in-vehicle terminal, a smart home appliance (e.g., a smart TV), an AR/VR device, and the like, but is not limited thereto. In one example, the terminal is configured with an AI model that can acquire the signals captured by the smart shirt as shown in FIG. 5 and FIG. 6 for processing.

Wherein, the server may also perform the AI-enhanced wearable photo-electro-tonoarteriography (PETAG) method provided by embodiments of the present invention. The server can be an independent physical server, a server cluster or a distributed system (e.g., a distributed cloud storage system) composed of multiple physical servers, or a cloud server that provides cloud computing services. For example, the terminal uploads the signal obtained from the smart shirt to the server, and the server completes processing of the signal.

In a feasible embodiment, the terminal as well as the server may be connected directly or indirectly by wired or wireless communication, and the present invention is not limited herein. For example, the terminal may send a data acquisition request to the server via the network.

In a feasible embodiment, the operating environment may also include a database, which may be used to store data such as TAG signals, heart disease information, etc.

The following is a specific description of the an AI-enhanced wearable photo-electro-tonoarteriography (PETAG) method provided in this embodiment.

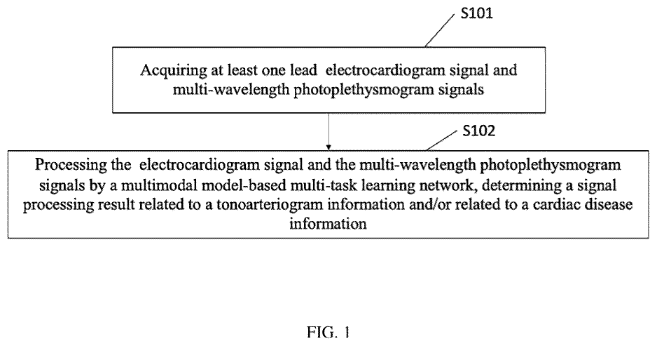

Specifically, as shown in FIG. 1, the method comprises the following steps S101-S102. S101: acquiring at least one lead ECG signal and multi-wavelength photoplethysmogram (MWPPG) signals; S102: processing the ECG signal and the MWPPG signals by a multimodal model-based multi-task learning network, determining a signal processing result related to a TAG information and/or related to a cardiac disease information.

Specifically, the present invention takes into account that limb II lead and chest V1 lead can be used for hypertension screening and to improve the identification of ECG abnormalities, such as atrial fibrillation; wherein, limb II lead and chest V1 lead are more exemplary for research in related directions compared to limb I lead; furthermore, considering that MWPPG signals can be used to estimate blood pressure, and that the time difference of each wavelength and its morphological information are all highly correlated with blood pressure information; therefore, in this embodiment, the ECG signal and MWPPG signal can be acquired through limb leads (e.g., leads I, II, and/or III) and chest lead VI.

Optionally, abnormal changes in cardiac electrical activity due to different cardiac diseases are usually reflected by changes in the 12-lead ECGs, so it is also possible to obtain additional cardiac information for the estimation of the TAG signal and its associated blood pressure information by acquiring the 12-lead ECG signals with the MWPPG signals.

Wherein, the multimodal model-based multi-task learning network (MMM-net) can be a deep neural network, and the processing of the MMM-net can reduce the overfitting of specific tasks and improve the adaptability and efficiency of different tasks. The embodiment of the present invention uses ECG signals and MWPPG signals obtained from different leads as inputs to the AI model, which can be processed by the neural network to estimate TAG signals, associated blood pressure information and heart disease information.

Wherein, said signal processing results associated with the TAG information include at least one of the TAG signal, systolic blood pressure, diastolic blood pressure, blood pressure variability and hypertension information; and said signal processing results associated with the heart disease information include at least one of the ECG, arrhythmia detection results and myocardial infarction detection results. Specifically, the signal processing results identified in this invention can be used to screen for and detect different heart diseases, including but not limited to arrhythmia, atrial fibrillation, and myocardial infarction.

The AI-enhanced wearable photo-electro-tonoarteriography (PETAG) method provided by embodiments of the present invention combines ECG signals acquired in specific two leads, particularly limb II lead and chest V1 lead, with multi-wavelength PPG signals for estimating TAG signals and their associated blood pressure information, and detecting atrial fibrillation, and the implementation of this solution can save computational costs and reduce manufacturing complexity without loss of accuracy. In addition, it is possible to combine the ECG signals acquired in specific leads such as 12 leads with the multi-wavelength PPG signals, which can be used to obtain additional cardiac information for estimating the TAG signal and its associated blood pressure information, screening and detecting different cardiac diseases, including but not limited to arrhythmia, atrial fibrillation, and myocardial infarction. In addition, ECG signals and information can be transmitted to other external electronic devices and cloud databases through wireless module.

The following is a description of the processing of the acquired signals as an embodiment.

In a possible embodiment, the step S101 of acquiring at least one lead ECG signal and MWPPG signals comprises the step A1:
A1: acquiring by collecting at least one lead ECG signal and MWPPG signals from a clothing worn by a target subject; wherein cloth of said clothing is provided with an ECG electrode and a multi-wavelength photoplethysmography sensors.

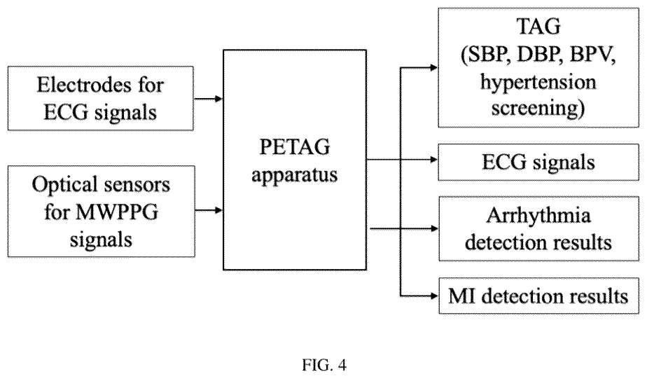

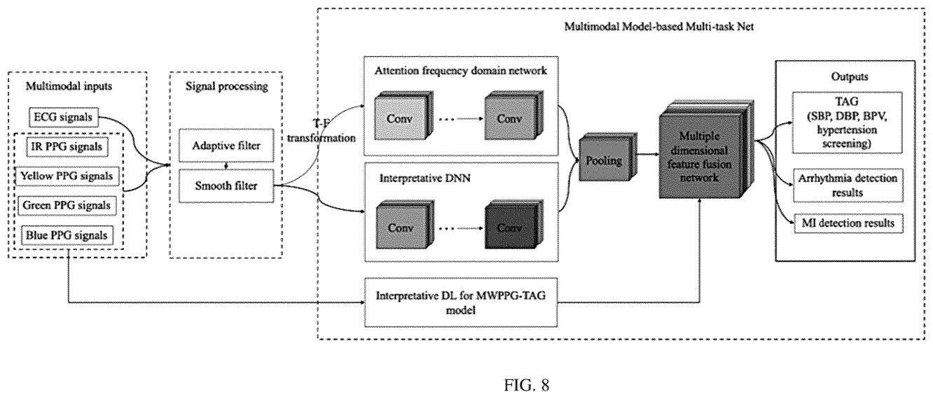

Specifically, as shown in FIG. 4, FIG. 5 and FIG. 6, the signals can be collected through the smart shirt worn by the test subject, and the collected signals includes an electrical signal (ECG signal) collected through an ECG electrode and a photoplethysmogram signal (e.g., multi-wavelength PPG signals) collected through an optical sensor. Preferably, as shown in FIG. 8, the photoplethysmogram signal may include at least one of the following: infrared PPG signal, yellow PPG signal, green PPG signal, blue PPG signal.

Wherein, the ECG electrode and the multi-wavelength PPG sensor are deployed in said clothing (e.g., smart shirt) in one of the following three forms.

The first form: the ECG electrode and the multi-wavelength photoplethysmography sensors are arranged at the clothing.

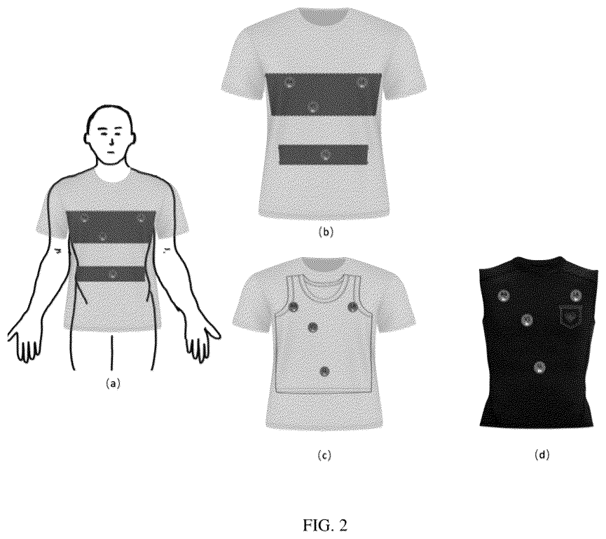

Specifically, as shown in (a) and (b) in FIG. 2, which illustrates the placement of ECG electrodes and multi-wavelength PPG sensors for acquiring multi-wavelength PPG signals from limb leads (which may be I, II, and/or III leads), and chest leads. Among them, the positions of the ECG electrodes and multi-wavelength PPG sensors are located on RA of the right arm, VI of the chest, and LA of the left arm, respectively, which are laid out from left to right in the tight band located in the chest; and the positions of the ECG electrodes and multi-wavelength PPG sensors for the LL of the left leg are in the tight band located in the waist.

The second form: manufacturing the ECG electrode and the multi-wavelength photoplethysmography sensors at a vest and/or a waistband combined with the electric conductive clothing.

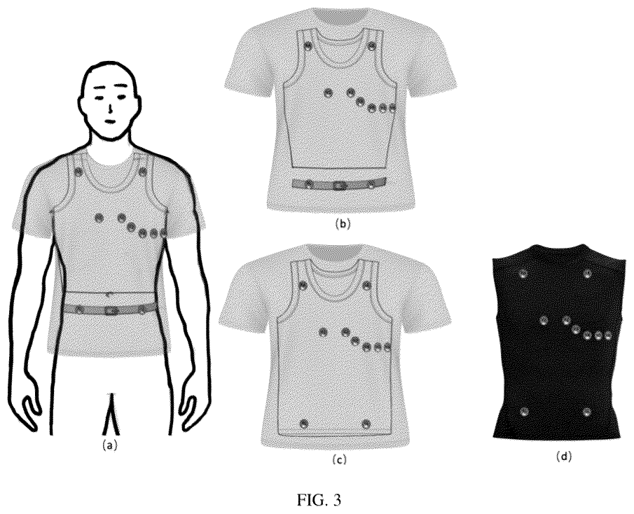

Specifically, as shown in (c) in FIG. 2 and in (a), (b), and (c) in FIG. 3. Therein, the ECG signals and multi-wavelength PPG signals in leads I, II, and/or III of the limb and VI of the chest are acquired in (c) in FIG. 2; the ECG signals and multi-wavelength PPG signals in 12 leads are acquired in (a), (b), and (c) in FIG. 3.

The third form: manufacturing the ECG electrode and the multi-wavelength photoplethysmography sensors at a modified tight clothing.

Specifically, as shown in (d) in FIG. 2 and in (d) in FIG. 3. Therein, the ECG signals and multi-wavelength PPG signals in leads I, II, and/or III of the limb and VI of the chest are acquired in (d) in FIG. 2; and the ECG signals and multi-wavelength PPG signals in lead 12 are acquired in (d) in FIG. 3.

In all three forms, the limb lead ECG may be placed according to the Mason-Likar system and the triangular apex position on the chest; the chest lead electrode placement conforms to the clinical ECG requirements to ensure a standardized signal for the clinician.

Wherein the EGC electrode is made of at least one of electronic fabric materials, ionic hydrogels and other soft electric conductive materials; the multi-wavelength photoplethysmography sensors is integrated in the ECG electrode.

The design associated with the smart shirt provided in this embodiment facilitates the repeatable applicability and comfort of the shirt.

The specific process of signal filtering processing in the present embodiment is described below in conjunction with the present embodiment.

In the present embodiment, considering that the noise present in the acquired signal may distort the signal and affect the accuracy of the signal processing result, the signal acquired in S101 is also filtered before performing the above-mentioned S102. Specifically, wherein before processing the ECG signal and the MWPPG signals by a multimodal model-based multi-task learning network, further comprising steps B1-B2:
B1: filtering noise from the ECG signal and the MWPPG signals to obtain a noise reduced signal; B11 iteratively performing the following operations until a stop iteration condition is met: combining impedance information between electrode of a left arm and a right arm and between electrode of a right arm and a left leg, filtering noise from the ECG signal and the MWPPG signals, and generating a current noise reduced signal; and using the noise reduced signal and the impedance information as input data for a next iteration, filtering the noise from the ECG signal and the MWPPG signals.
B2: converting the noise reduced signal to obtain a noise reduced ECG signal and MWPPG signals of different leads.

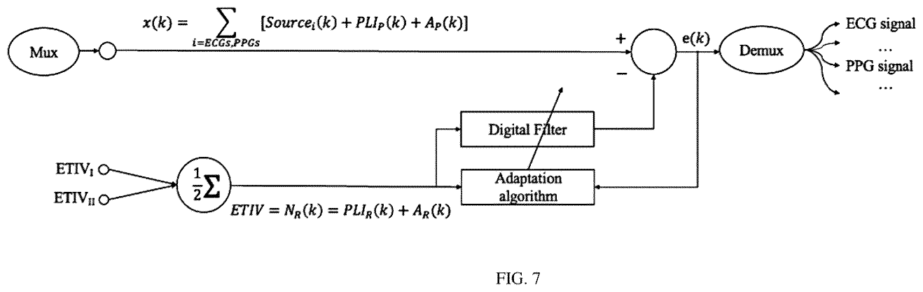

Specifically, during the acquisition of ECG and multi-wavelength PPG signals, the human body is susceptible to relative displacement of electrodes/sensors and skin as well as stretching of the skin under dynamic conditions, resulting in changes in electrode-skin impedance (ETIV), which causes motion artifact (MA). MA is the largest source of noise in wearable physiological signals monitoring, which can distort the signal and its analysis, or even cause misclassification and threaten the patient's life. Therefore, reducing this type of noise to obtain high quality signals is critical for wearable signals and systems. Among other things, skin strain causes changes in skin impedance, resulting in changes in the electrical potential between 2 electrodes, reflecting the motion trend. Therefore, the motion artifact signal can be qualitatively described by measuring ETIV. FIG. 7 shows the adaptive filter (adaptive algorithm) for suppressing MA and power line interference (PLI) in ECG signals and multi-wavelength PPG signals. The main inputs to the adaptive filter are the ECG signal and the multi-wavelength PPG signal with MA and PLI, x(k), which are processed by the multiplexing unit as a single channel signal (i.e., multiple signals as a single channel with the same input signal, shown as Mux in FIG. 7) with reference to Eq (1) below. The average value of ETIVI and ETIVII is used as the reference input N_{R}(k) for PLI and MA related noise, referring to Eq (2) below. By iterative operation, the adaptive filter can automatically reduce the noise in the corrupted signal to generate the noise reduction signal e(k). After obtaining e(k), the splitting unit reconverts the signal into an independent signal (shown as Demux in FIG. 7) to obtain the noise-reduced ECG signal and the multi-wavelength PPG signal for different leads for further analysis. After adaptive filtering, the signals are smoothed and filtered to reduce the abrupt changes in the signals for better extraction of the characteristic parameters.

$$x ( k ) = {\sum }_{i = ECGs , PPGs} {[ Source}_{i} ( k ) + {PLI}_{P} ( k ) + {A}_{R} ( k ) ] \# \qquad ( 1 )$$

In the above Eq (1), x(k) is the input ECG signals and multi-wavelength PPG signals with MA and PLI interference, where Sourceᵢ(k) is the ECG signals and PPG signals; PLIₚ(k) is the power line interference; and A_{R}(k) is the motion artifact.

ETIV=N_{R}(k)=PLI_{R}(k)+A_{R}(k) #(2)

In the above Eq (2), ETIV is the average value of ETIVI and ETIVII; PLI_{R} (k) is the power interference; A_{R} (k) is the motion artifact.

Optionally, the stop iteration conditions may comprise the current number of iterations reaching a preset number of iterations, and the percentage of noise contained in the output noise reduction signal reaching a preset requirement, etc.

The following is a description of the specific process of signal processing in this embodiment.

In a possible embodiment, S102: processing the ECG signal and the multi-wavelength signal by a multimodal model-based multi-task learning network, determining a signal processing result related to a TAG information and/or related to a cardiac disease information, comprising the following steps C1-C4:
C1: performing feature extraction on the ECG signal and the MWPPG signals through frequency domain attention based neural network and time domain interpretative based neural network to obtain a first feature information;
C2: performing feature extraction on the MWPPG signals to obtain a second feature information;
C3: performing pooling operation on the first feature information;
C4: performing feature fusion classification process against the second feature information and the pooled first feature information to determine the signal processing result related to the TAG information and/or related to the cardiac disease information.

Specifically, as shown in FIG. 8, the AI model referred to in FIGS. 5 and 6 is trained by a deep neural network with a multimodal model-based multi-task learning network (MMM-net), and feature extraction is performed by an interpretable deep neural network (IDNN) architecture with frequency-domain attention-based and time-domain-based features to obtain the first feature information; the first feature information is pooled by a pooling layer to perform a pooling operation. The pooled first feature information is obtained (as shown in FIG. 8, the frequency-domain attention neural network can be used for Cony operations on ECG and multi-wavelength PPG signals to obtain feature information A; the interpretable deep neural network is used to Cony operations for ECG and multi-wavelength PPG signals to obtain feature information B; feature information A and feature information B are input to the pooling layer); The pooled first feature information can be fused with the second feature information extracted from the multi-wavelength PPG signals to perform feature fusion classification through a multi-dimensional feature fusion network. Among them, the extraction of the second feature information can be implemented by a machine learning based MWPPG-TAG model, which preferably can be a deep learning neural machine learning network based on interpretability; accordingly, other machine learning networks can also be used for feature extraction, which is not limited by this invention.

In the above operation, the MMM-net reduces task-specific overfitting and improves adaptability and efficiency for different tasks, where the frequency-domain attention-based multilayer convolutional neural network and the time-domain interpretability-based deep multilayer convolutional neural network enhance the ability of the model to focus on important time-and frequency-domain features. The features are extracted by a hybrid model combining data-driven and model-driven. The feature vectors are fed into a multidimensional feature fusion network for classification and fusion for the output of TAG signals. Blood pressure-related information includes systolic blood pressure, diastolic blood pressure, blood pressure variability, hypertension screening information, and cardiac disease detection information, where cardiac diseases include but are not limited to arrhythmia, atrial fibrillation and myocardial infarction.

The ECG signals obtained from different specific leads and multi-wavelength PPG signals obtained from multi-wavelength optical sensors are used as inputs to the AI model. After the neural network chip or signal processing unit, the TAG signals and related blood pressure information can be estimated. The ECG signals and specific cardiac abnormal diseases can be detected. The combination of the ECG signal acquired from specific two leads especially limb II lead and chest V1 lead, and multi-wavelength PPG signals can be used to estimate the TAG signal and its related blood pressure information, ECG signal and atrial fibrillation detection results. Combining ECG signals acquired from specific leads such as 12 leads with multi-wavelength PPG signals can be used to obtain additional cardiac information for estimating TAG signals and its associated blood pressure information, ECG signals, screening and detecting different cardiac diseases including but not limited to arrhythmias, atrial fibrillation and myocardial infarction.

In a possible embodiment, the AI-enhanced wearable photo-electro-tonoarteriography (PETAG) method provided by embodiments of the present invention further comprises at least one of steps D1-D2,
D1: transmitting the signal processing result to a user apparatus to display the signal processing result to a user; the user apparatus comprises at least one of a mobile phone, a watch and glasses;
D2: uploading the signal processing result to a cloud database and/or a medical platform.

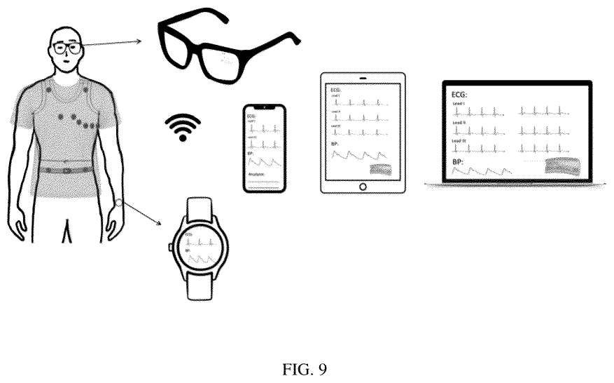

Specifically, as shown in FIG. 9, the signals acquired by the smart shirt (signals acquired in S101) can be communicated with at least one external electronic device via a wireless module, and these signals can be transmitted to electronic devices (e.g., cell phones, tablets, computers, and other devices) for further processing and display. In addition, the signal processing results (results obtained in S102) can be transmitted to an external wearable device, such as a watch, glasses, cell phone, etc., for display; or uploaded to a cloud database and telemedicine platform for continuous recording of signal processing results (e.g., TAG information, heart disease information, etc.) determined by the test subject based on the acquired signals.

In an embodiment of the present invention, there is also provided an AI-enhanced wearable photo-electro-tonoarteriography (PETAG) system based on PPG signals and ECG signals. Specifically, comprising clothing and an electronic device equipped with a sensing module and a communication module; wherein the said clothing acquires ECG from at least one lead and multi-wavelength PPG signals via the sensing module and transmits the acquired ECG and multi-wavelength PPG signals to the said electronic device via the communication module; the said electronic device performs the steps associated with the AI-enhanced wearable photo-electro-tonoarteriography (PETAG) method provided in the above embodiment.

Specifically, the structural unit laid out in the clothing may be referred to the schematic diagram of the smart shirt referred to in FIGS. 2 and 3; wherein the type of smart shirt can be but is not limited to a vest, short sleeve or long sleeve shirt. The specific deployment may also be referred to the relevant description of the embodiment corresponding to step A1 above. The said sensing module includes a ECG electrode and multi-wavelength PPG sensors (as shown in FIG. 4). Optionally, as shown in FIG. 6, the smart shirt is also provided with an analog front end (AFE) module and a processing control module connected to the AFE module via ADC; the processing control module includes an MCU and a wireless module; the smart shirt can be communicatively connected to the wireless module in the electronic device via the wireless module and transmit the collected signals to the electronic device. Optionally, as shown in FIG. 5, the smart shirt is also equipped with an AFE module and an MCU connected to the AFE module via ADC, and then the MCU is connected to the wireless module and the power supply module, respectively; the smart shirt can transmit the collected signals to the electronic device (specifically, communicating with the filter) via the wireless module.

In one example, the system structure shown in FIG. 5 and FIG. 6 can be adjusted with reference to each other, such that the smart shirt shown in FIG. 6 can cooperate with the external electronic device shown in FIG. 5 to form another system structure.

It should be noted that in the optional embodiment of this invention, the data involved (such as ECG signal, multi-wavelength PPG signals, TAG signal) and other related data. When the above embodiment of this invention is applied to a specific product or technology, it requires permission or consent from the object of use, and the collection, use and processing of the related data need to comply with the relevant laws, regulations and standards of the relevant countries and regions. In other words, if the data related to the object in this embodiment is involved, the data needs to be obtained with the authorization and consent of the object and in accordance with the relevant laws, regulations and standards of the country and region.

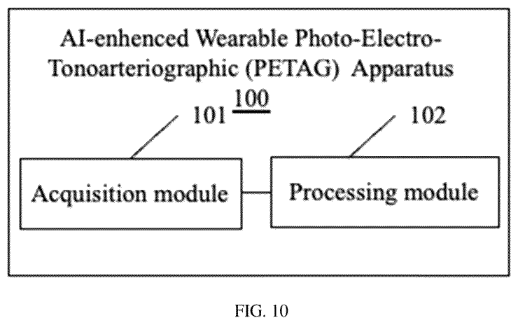

Embodiments of the present invention provide an AI-enhanced wearable photo-electro-tonoarteriography (PETAG) apparatus as shown in FIG. 10. The AI-enhanced wearable photo-electro-tonoarteriography (PETAG) apparatus 100 based on PPG and ECG may include: an acquisition module 101, a processing module 102.

Wherein, the acquisition module 101 for acquiring at least one lead ECG signal and MWPPG signals; the processing module 102 for processing the ECG signal and the MWPPG signals by a multimodal model-based multi-task learning network, determining a signal processing result related to a TAG information and/or related to a cardiac disease information.

In a possible embodiment, the acquisition module 101 acquires at least one lead EGC signal and MWPPG signals comprises acquiring by collecting at least one lead ECG signal and MWPPG signals from a clothing worn by a target subject; wherein cloth of said clothing is provided with an ECG electrode and a multi-wavelength photoplethysmography sensors.

In a possible embodiment, the ECG electrode and the multi-wavelength photoplethysmography sensors are arranged at the clothing using one of the following: manufacturing the ECG electrode and the multi-wavelength photoplethysmography sensors at a chest portion and a waist portion of a detachable tight belt, with the detachable tight belt being fixed at a corresponding position of an electric conductive clothing; manufacturing the ECG electrode and the multi-wavelength photoplethysmography sensors at a vest and/or a waistband combined with the electric conductive clothing; manufacturing the ECG electrode and the multi-wavelength photoplethysmography sensors at a modified tight clothing.

In a possible embodiment, the EGC electrode is made of at least one of electronic fabric materials, ionic hydrogels and other soft electric conductive materials; the multi-wavelength photoplethysmography sensors is integrated in the ECG electrode.

In a possible embodiment, before processing the ECG signal and the MWPPG signals by a multimodal model-based multi-task learning network, further comprising: filtering noise from the ECG signal and the MWPPG signals to obtain a noise reduced signal; converting the noise reduced signal to obtain a noise reduced ECG signal and MWPPG signals of different leads.

In a possible embodiment, the filtering noise from the ECG signal and the MWPPG signals to obtain the noise reduced signal specifically used in: iteratively performing the following operations until a stop iteration condition is met; combining impedance information between electrode of a left arm and a right arm and between electrode of a right arm and a left leg, filtering noise from the ECG signal and the MWPPG signals, and generating a current noise reduced signal; and using the noise reduced signal and the impedance information as input data for a next iteration, filtering the noise from the ECG signal and the MWPPG signals.

In a possible embodiment, the processing module 102 processes the ECG signal and the multi-wavelength signal by a multimodal model-based multi-task learning network, determining a signal processing result related to a TAG information and/or related to a cardiac disease information, specifically used in: performing feature extraction on the ECG signal and the MWPPG signals through frequency domain attention based neural network and time domain interpretative based neural network to obtain a first feature information; performing feature extraction on the MWPPG signals to obtain a second feature information; performing pooling operation on the first feature information; performing feature fusion classification process against the second feature information and the pooled first feature information to determine the signal processing result related to the TAG information and/or related to the cardiac disease information.

In a possible embodiment, the signal processing result related to the TAG information comprises at least one of a TAG signal, a systolic blood pressure information, a diastolic blood pressure information, a blood pressure variation information and a hypertension information; the signal processing result related to the cardiac disease information comprises at least one of an electrocardiogram, an arrhythmia detection result and a myocardial infarction detection result.

In a possible embodiment, further comprising a transmission module, the transmission module for performing at least one of the following: transmitting the signal processing result to a user apparatus to display the signal processing result to a user; the user apparatus comprises at least one of a mobile phone, a watch and glasses; uploading the signal processing result to a cloud database and/or a medical platform.

The device of the present invention embodiment can perform the method provided in the present invention embodiment with similar implementation principles. The actions performed by the modules in the device of the present invention are corresponding to the steps in the method of the present invention, and the detailed functional description of the modules of the device can be specifically referred to the description in the corresponding method shown in the previous section, which will not be repeated here.

The processing results, target signals, ECG signals, multi-wavelength PPG signals, etc. involved in the embodiments of the present invention can be stored by blockchain technology. The blockchain referred to in this invention is a new application model of computer technology such as distributed data storage, peer-to-peer transmission, consensus mechanism, and encryption algorithm. A blockchain, essentially a decentralized database, is a string of data blocks generated using cryptographic methods associated with each block containing a certain amount of processed data for verifying the validity of its information (forgery-proof) and generating the next block. Blockchain can include a blockchain underlying platform, a platform product service layer, and an application service layer.

Provided in this invention is an electronic apparatus comprising a memory, a processor, and a computer program stored on the memory, the processor executing the above computer program to implement the steps of the AI-enhanced wearable photo-electro-tonoarteriography (PETAG) method, which can be achieved in comparison to related technologies: specifically, after acquiring the ECG signal of a specific lead and PPG signal, it can be input to a multimodal model-based multi-task learning network for signal processing to determine the signal processing results related to TAG information and/or related to heart disease information. The implementation of this application can save computational costs and reduce the complexity of manufacturing detection devices related to blood pressure, and also ensure the accuracy of signal processing.

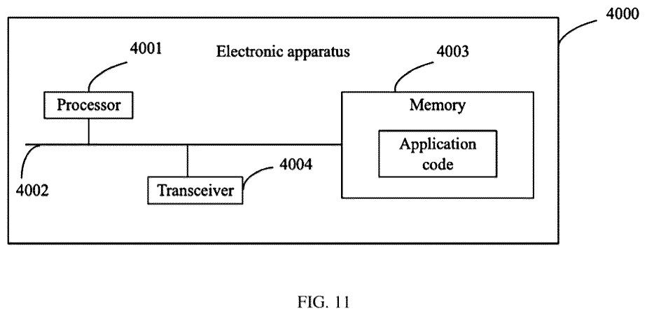

In an optional embodiment an electronic apparatus is provided, as shown in FIG. 11, wherein the electronic apparatus 4000 shown in FIG. 11 includes: a processor 4001 and a memory 4003. wherein the processor 4001 and the memory 4003 are connected, e.g., via a bus 4002. Optionally, the electronic apparatus 4000 may also include a transceiver 4004, which may be used for data interaction between this electronic apparatus and other electronic devices, such as the sending of data and/or the receiving of data, etc. It should be noted that the transceiver 4004 is not limited to one in practical applications, and the structure of the electronic apparatus 4000 does not constitute a limitation of this invention.

The processor 4001 may be a CPU (Central Processing Unit), a general-purpose processor, a DSP (Digital Signal Processor), FPGA (Field Programmable Gate Array) or other programmable logic device, transistorized logic device, hardware component, or any combination thereof. It may implement or execute various exemplary logic boxes, modules, and circuits described in conjunction with the disclosure of this invention. Processor 4001 may also be a combination that implements a computing function, such as a combination containing one or more microprocessors, a combination of a DSP and a microprocessor, etc.

The bus 4002 may include a pathway to transfer information between the above components. Bus 4002 may be a PCI (Peripheral Component Interconnect) bus or an EISA (Extended Industry Standard Architecture) bus, for example. Bus 4002 can be divided into address bus, data bus, control bus, etc. For the convenience of representation, only a thick line is used in FIG. 11, but it does not mean that there is only one bus or one type of bus.

Memory 4003 can be ROM (Read Only Memory) or other types of static storage devices that can store static information and instructions, RAM (Random Access Memory) or other types of dynamic storage devices that can store information and instructions, or EEPROM (Electrically Erasable Programmable Read Only Memory), CD-ROM (Compact Disc Read Only Memory) or other optical disc storage, optical disc storage (including compressed disc, laser disc, optical disc, or other optical disc storage, optical disc storage (including compact disc, laser disc, optical disc, digital universal disc, Blu-ray disc, etc.), disk storage media, other magnetic storage devices, or any other media capable of being used to carry or store computer programs and capable of being read by a computer, without limitation herein.

Memory 4003 is used to store a computer program for executing an embodiment of the present invention and is controlled for execution by processor 4001. The processor 4001 is used to execute the computer program stored in the memory 4003 to implement the steps shown in the preceding method embodiment.

Wherein, the electronic apparatus includes, but is not limited to: a server, a terminal.

Embodiments of the present invention provide a computer readable storage medium having a computer program stored on the computer readable storage medium, the computer program being executed by the processor to implement the steps and corresponding contents of the preceding method embodiments.

Embodiments of the present invention also provide a computer program product comprising a computer program. The computer program can realize the steps and corresponding contents of the preceding method embodiments. when executed by a processor.

The terms “first”, “second”, “third”, “fourth”, “third” and “fourth” in the specification and claims of this invention and in the accompanying drawings above”, “1”, “2”, etc. (if present) are used to distinguish similar objects and need not be used to describe a particular order or sequence. It should be understood that the used data is interchangeable so that embodiments of the present invention described herein can be implemented in an order other than that illustrated or described in the text.

It should be understood that while the flow diagrams of embodiments of the present invention indicate the individual operational steps by arrows, the order in which these steps are performed is not limited to the order indicated by the arrows. Unless explicitly stated herein, in some implementation scenarios of embodiments of the present invention, the implementation steps in the respective flowcharts may be performed in other orders as desired. In addition, some or all of the steps in each flowchart may include multiple sub-steps or multiple stages based on actual implementation scenarios. Some or all of these sub-steps or phases may be executed at the same moment, and each of these sub-steps or phases may also be executed separately at different moments. In scenarios where the execution time is different, the order of execution of these sub-steps or stages can be flexibly configured according to the needs, and this invention embodiment is not limited in this regard.

It should be noted that for a person of ordinary skill in the art, other similar means of implementation based on the technical idea of the present invention, without departing from the technical idea of the present invention, also fall within the scope of protection of the embodiments of the present invention.

1. An AI-enhanced wearable photo-electro-tonoarteriography (PETAG) method and apparatus, comprising:
acquiring at least one lead electrocardiogram signal and multi-wavelength photoplethysmogram (MWPPG) signals;
processing the electrocardiogram signal and the MWPPG signals by a multimodal model-based multi-task learning network, determining a signal processing result related to a TAG information and/or related to a cardiac disease information.

2. The method according to claim 1, wherein the step of acquiring at least one lead EGC signal and MWPPG signals comprises:
acquiring by collecting at least one lead electrocardiogram signal and MWPPG signals from a clothing worn by a target subject;
wherein cloth of said clothing is provided with an electrocardiogram signal and a multi-wavelength photoplethysmography sensors.

3. The method according to claim 2, wherein the ECG electrode and the multi-wavelength photoplethysmography sensors are arranged at the clothing using one of the following:
manufacturing the ECG electrode and the multi-wavelength photoplethysmography sensors at a chest portion and a waist portion of a detachable tight belt, with the detachable tight belt being fixed at a corresponding position of an electric conductive clothing;
manufacturing the ECG electrode and the multi-wavelength photoplethysmography sensors at a vest and/or a waistband combined with the electric conductive clothing;
manufacturing the ECG electrode and the multi-wavelength photoplethysmography sensors at a modified tight clothing.

4. The method according to claim 2, wherein the EGC electrode is made of at least one of electronic fabric materials, ionic hydrogels and other soft electric conductive materials; the multi-wavelength photoplethysmography sensors is integrated in the ECG electrode.

5. The method according to claim 1, wherein before processing the electrocardiogram signal and the MWPPG signals by a multimodal model-based multi-task learning network, further comprising:
filtering noise from the electrocardiogram signal and the MWPPG signals to obtain a noise reduced signal;
converting the noise reduced signal to obtain a noise reduced electrocardiogram signal and MWPPG signals of different leads.

6. The method according to claim 5, wherein the filtering noise from the electrocardiogram signal and the MWPPG signals to obtain the noise reduced signal comprises:
iteratively performing the following operations until a stop iteration condition is met:
combining impedance information between electrode of a left arm and a right arm and between electrode of a right arm and a left leg, filtering noise from the electrocardiogram signal and the MWPPG signals, and generating a current noise reduced signal; and using the noise reduced signal and the impedance information as input data for a next iteration, filtering the noise from the electrocardiogram signal and the MWPPG signals.

7. The method according to claim 1, wherein the step of processing the electrocardiogram signal and the multi-wavelength signal by a multimodal model-based multi-task learning network, determining a signal processing result related to a TAG information and/or related to a cardiac disease information, comprising:
performing feature extraction on the electrocardiogram signal and the MWPPG signals through frequency domain attention based neural network and time domain interpretative based neural network to obtain a first feature information;
performing feature extraction on the MWPPG signals to obtain a second feature information;
performing pooling operation on the first feature information;
performing feature fusion classification process against the second feature information and the pooled first feature information to determine the signal processing result related to the TAG information and/or related to the cardiac disease information.

8. The method according to claim 1, wherein the signal processing result related to the TAG information comprises at least one of a TAG signal, a systolic blood pressure information, a diastolic blood pressure information, a blood pressure variation information and a hypertension information;
the signal processing result related to the cardiac disease information comprises at least one of an electrocardiogram, an arrhythmia detection result and a myocardial infarction detection result.

9. The method according to claim 1, further comprising at least one of the following:
transmitting the signal processing result to a user apparatus to display the signal processing result to a user; the user apparatus comprises at least one of a mobile phone, a watch and glasses;
uploading the signal processing result to a cloud database and/or a medical platform.

10. An AI-enhanced wearable photo-electro-tonoarteriography (PETAG) apparatus , comprising:
an acquisition module for acquiring at least one lead electrocardiogram signal and MWPPG signals;
a processing module for processing the electrocardiogram signal and the MWPPG signals by a multimodal model-based multi-task learning network, determining a signal processing result related to a TAG information and/or related to a cardiac disease information.

11. An AI-enhanced wearable photo-electro-tonoarteriography (PETAG) system, comprising the said clothing and electronic devices designed with sensing module and communication module;
acquiring by collecting at least one lead electrocardiogram signal and MWPPG signals from the sensing module in the clothing, and transmitting the obtained electrocardiogram signal and MWPPG signals to the said electronic devices through the communication module;
the said electronic devices perform the method steps of claim 1.

12. The system according to claim 11, characterized in that said sensing module comprises an ECG electrode and a multi-wavelength photoplethysmography sensors.
